(19) United States
(12) Patent Application Publication
(10) Pub. No.: US 2007/0038233 A1
(43) Pub. Date: Feb. 15, 2007

(54) Surgical clips, systems and methods thereof

(75) Inventors: Jaime Martinez, Pompton Plains, NJ (US); Clem Darling III, Delmar, NY (US)

Correspondence Address: GIBBONS, DEL DEO, DOLAN, GRIFFINGER & VECCHIONE, 1 RIVERFRONT PLAZA, NEWARK, NJ, 07102-5497, US

(73) Assignee: Medical Device Concepts LLC

(21) Appl. No.: 11/201,870

(22) Filed: Aug. 11, 2005

(51) Int. Cl. A61B 17/08

(52) U.S. Cl. 606/157

(57) ABSTRACT

A surgical clip that is used to temporary occlude a blood vessel of a patient. The clip has a pair of longitudinally extending members that are spring biased to a closed position to squeeze therebetween the blood vessel of the patient. The members have a first region adjacent the biasing spring and extend to mating clamping surface regions where the blood vessel is positioned. There is an angular offset between the first region and the clamping surface regions for convenience in positioning the surgical clip. In another embodiment, there is a L-shaped surgical clip that is dimensioned so as to temporary occlude a plurality of vessels simultaneously. There is also a container that is customized to provide certain sizes and quantities of surgical clips for a particular surgical procedure and a method of determining those clips for the convenience of the surgeon in carrying out the procedure.

### FIELD OF THE INVENTION

The present invention relates generally to surgical devices for temporarily occluding a blood vessel during an operation, and, more particularly, to a surgical clip that has added convenience and versatility in its use in temporarily occluding a blood vessel.

### BACKGROUND OF THE INVENTION

Surgical clips are used in a relatively wide variety of medical operations, particularly surgical procedures undertaken by vascular surgeons in the treatment of cardiovascular disease and some of those procedures include carotid endarectomies and femoral poplitealbypass surgeries. In those, and other such surgical procedures, it is necessary for the vascular surgeon to temporarily occlude certain target blood vessels, thereby stopping the blood flow through such vessels so that the vessels can, for example, be cleared of plaque or other blockages that could prevent proper blood flow in the patient.

Thus, to carry out that temporary occlusion of such blood vessel, a variety of devices are utilized by the surgeon and, among those devices are vessel clamps or clips which, when used with or without sutures, are effective in temporarily stopping or limiting blood flow for a period of time necessary for the surgeon to carry out and complete other aspects of the surgical technique.

One typical type of surgical clip includes a coiled spring at the proximal end and having two longitudinally extending members leading outwardly from the spring to distal ends. The spring biases the distal ends toward each other such that the distal ends are normally in a closed position where they abut against each other. The user can move the members against the bias of the spring so as to open the distal ends and move them away from each other. With this type of clip, the distal ends are positioned by the surgeon so as to surround the blood vessel to be occluded while the distal ends are in the open position and the surgeon can simply release the members that enables the spring to move the distal ends to the closed position where they are again adjacent to each other pinching the blood vessel therebetween. As such, that type of surgical clip is a relatively simple yet effective device for occluding a blood vessel.

There are, however, a number of drawbacks with that type of surgical clip. First of all the present designs are constructed of straight members from the proximal or spring end to the distal end and, therefore, they lack versatility in the ability to be introduced into the patient and reach the particular blood vessel that is desired be occluded, particularly where the blood vessel is not readily accessible and there are other instruments being used on patient that make the use of a straight surgical clip difficult to use. Next, the spring bias is relatively constant among the differing surgical clips and there are times when it would be advantageous to have available to the surgeon, a surgical clip with differing bias. As a further drawback, the present surgical clips are not suitable for occluding more than one blood vessel at a time and, again, there are times where it would be advantageous to have a surgical clip that was capable of occluding at least two blood vessels simultaneously with the same clip.

As a further difficulty with the clips, themselves, they are normally provided to the surgeon in a relatively unorganized manner that is, the clips are conventionally simply secured to a crude cardboard holder in sterilized condition but presented in a random form. Since 4-5 surgical clips can be used during a normal surgery, the random nature makes it difficult to select and use the particular clips that are needed in a specific operation and it would be advantageous to have a tray where the clips are organized and arranged for a specific surgery for the convenience of the surgeon to be able to more easily select and use the correct clip.

Accordingly, there is, therefore, a need for a surgical clip that overcomes the aforedescribed difficulties with present surgical clips and for a system that provides an organized access to surgical clips that customizes the types and quantities of such clips for a particular surgical procedure.

### SUMMARY OF THE INVENTION

The present invention relates to a surgical clip for use in temporarily occluding one or more blood vessels during a surgical procedure and to a system of providing such surgical clips in a convenient, organized fashion that is customized for a particular procedure.

In one embodiment of the present invention, the surgical clip comprises a pair of longitudinally extending members that have distal and proximal ends. At or proximate to the distal ends of both of the longitudinal extending members, there is formed a clamping surface region and each of the longitudinal extending member also has a first region where there is a biasing element that biases the clamping surface regions of the longitudinally extending members together in a closed position where those mating clamping surface regions are in an abutting relationship. The clamping surface region of the longitudinal extending members extend outwardly at a less than orthogonal angle with respect to the first regions in order to enable the surgeon to effectively utilize the surgical clip to occlude blood vessels that are relatively inaccessible.

As such, with the longitudinal extending members formed with an offset intermediate the first regions to the clamping surface regions, the configuration allows for greater ease of use as access, insertion and application of the surgical clip to the vessels is more efficient. In addition, the offset allows the insertion of the surgical clip to avoid entanglement of sutures if sutures are being used in conjunction with the surgical clip.

As a further feature of the present invention, the closing pressure, that is, the spring pressure that biases the clamping surface regions of the longitudinal extending members toward their closed position can be variable rather than a constant compression pressure so that the surgeon can utilize a surgical clip that has the closing pressure particularly suited and selected for accommodating the condition of the various vessels to be occluded. The variable pressure feature can be accomplished in a number of ways, such as by altering the spring mechanism that provides the closing bias, modifying the cross-section of the clip geometry to minimize contact stresses at the clamping surface regions where the vessel is clamped, adding a protective sleeve or by manufacturing the surgical clip out of a pliable material such as a polycarbonate plastic. If a protective coating or plastic material is used, the surgical clip would then be a single use device to avoid subjecting the plastic material to multiple sterilization processes.

An additional feature of the present invention can be with the use of color coding of the individual surgical clips or sets of clips, that is, the surgical clips can be color coded in accordance with one or more of their closing pressure, size, type and use. Thus, a surgical clip having a particular closing pressure can therefore be a common color to readily identify to the surgeon that closing pressure and the same color coding can also be used to identify the size of a particular clip such as having all small clips of the same color so that the surgeon can quickly identify the desired surgical clip. The color coding principle can also be applied to sets of surgical clips where a set, for example, of surgical clips to be used for a carotid endarectomy could all be the same color so that the surgeon could immediately recognize the set of surgical clips intended and customized for that specific procedure.

A further innovative feature of the present inventive surgical clip is in the design of a dual or multi-vessel surgical clip such that there is a large L-shaped configuration that has the ability of securing a large diameter vessel and, at the same time, also secure one of more additional vessels stemming from the larger blood vessel. The feature of a dual or multi vessel clip provides the benefit of ease in use so as to reduce surgical time and also eliminates a high of clutter and confusion resulting from 5 or 6 clips used at the same surgical site.

As a still further feature of the present invention, there is a surgical clip system comprising a container that has a plurality of receptacles for receipt of a surgical clip. The system customized the receptacles to include clips of at least one predetermined type and number in accordance in accordance with a particular surgical operation. By use of the system, a tray can be provided to the surgeon that has a certain number and sizes of vascular lips that are specially selected to be used during a specific operation so that the surgeon has the correct surgical clips available for the operation being carried out and the surgeon can readily select the proper surgical clip without the need to search through a number of extraneous surgical clips that may not even be usable in carrying out that operation. As examples, if the operation is a femoral popliteal bypass, there may be at least four clips of a particular size and another four clips of a smaller size. If the operation is an a carotid endarectomy, there may be at least two clips of a particular size And at least two other clips of a smaller size. As can thereof be seen, the tray is customized and has receptacles that receive the particular surgical clips that are used during a specific surgical procedure.

These and other features and advantages of the present invention will become more readily apparent during the following detailed description taken in conjunction with the drawings herein.

### BRIEF DESCRIPTION OF THE DRAWINGS

FIG. 1 is a side view of a typical prior art surgical clip;

FIGS. 2A-2F are side views of various surgical clips constructed in accordance with the present invention;

FIGS. 3A-3D are perspective views of various surgical clips generally corresponding to the surgical clips shown in FIGS. 2A-2D;

FIG. 4A, 4B and 4C are a top view, a side views and a perspective view of a multi-vessel surgical clip of the present invention;

FIG. 5 is a schematic view of the multi-vessel surgical clip of FIGS. 4A-4C in an operative position;

FIG. 6 is a top view of a backing supporting a plurality of various surgical clips;

FIG. 7A and 7B are top views of containers constructed in accordance with the present invention and retaining certain pre-selected surgical clips.

### DETAILED DESCRIPTION OF THE INVENTION

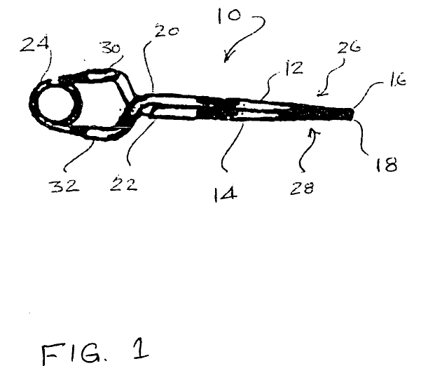

Referring now to FIG. 1, there is shown a side view of a typical surgical clip 10 that is currently in use in carrying out surgical procedures. As can be seen, the surgical clip 10 has a pair of longitudinally extending members 12, 14 each of which has a distal end 16, 18 respectively and proximal ends 20, 22. At the proximal ends 20, 22 there is a biasing element in the form of a coiled spring 24 that provides a bias tending to draw the distal ends 16, 18 toward each other into the closed position as illustrated in FIG. 1. At or proximate to the distal ends there is located clamping surface regions 26, 28 where the particular blood vessel is actually trapped between the longitudinally extending members 12, 14 to occlude that vessel. The coiled spring 24 includes actuating legs 30, 32 where the user can push inwardly on the actuating legs 30, 32 and separate the clamping surface regions 26, 28 against the bias of the coiled spring 24.

As such, the surgeon can simply push inwardly on the actuating legs 30, 32 to separate the clamping surface regions 26, 28 to an open position, locate the blood vessel to be occluded within the spaced apart clamping surface regions 26, 28 and then release the pressure on the actuating legs 30, 32 to bring the mating clamping surface regions 26, 28 together such that the bias of the coiled spring 24 clamps about the blood vessel to occlude that vessel. The surgical clip 10 presently in use is provided to the surgeon in various lengths for use in reaching and occluding a particular blood vessel depending upon its size and location. As can be seen, the configuration of the longitudinally extending members 12, 14 is constant or smooth from the distal ends 16, 18 to the proximal ends 20, 22 although there may be a curvature to longitudinally extending members 12, 14 along their length.

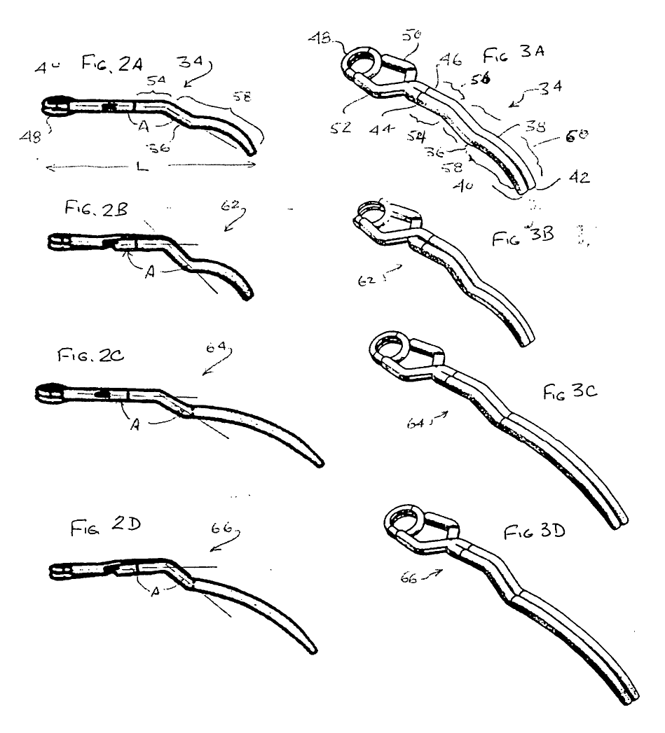

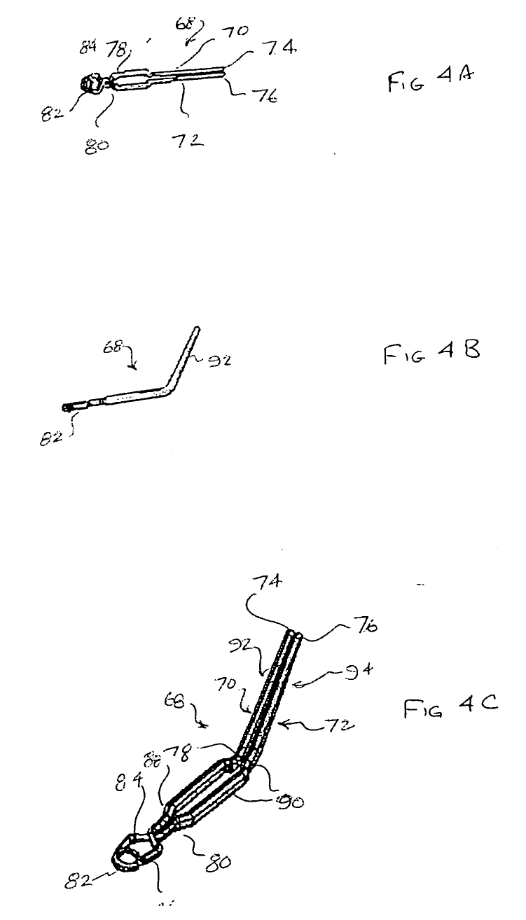

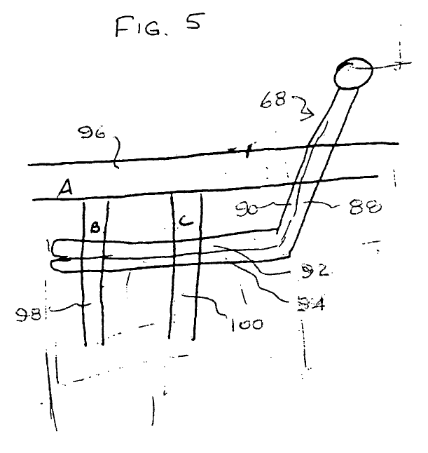

Turning now to FIGS. 2A-D and FIGS. 3A-D there is shown, side views and perspective views, respectively, of various surgical clips constructed in accordance with the present invention. Accordingly, taking FIGS. 2A and 3A initially, there can be seen a surgical clip 34 that includes a pair of longitudinally extending members 36, 38 with both having distal ends 40, 42 and proximal ends 44, 46. At the proximal ends 44, 46, there is a coiled spring 48 and a pair of actuating legs 50, 52. While a coiled spring is shown, it is noted that other biasing means can be used to provide the desired bias on the longitudinally extending members 36, 38, including, but not limited to, a coil or leaf spring, materials having inherent flexibility or memories, and the use of elastomers such as a flexible sleeve that encircle the extending members of the clip. At or proximate to the proximal ends 44, 46 each of the longitudinally extending members 36, 38 has a first region 54, 56 extending from the coiled spring 48 that is a generally straight configuration and there are also clamping surface regions 58, 60 that extend from the first regions 54, 56 and when the vascular vessel is located in occluding the vessel during the use of the surgical clip 34.

As can now be seen, the mating clamping surface regions 58, 60 extend from the first regions 54, 56 at a less than orthogonal offset angle A, that is, less than 90 degrees. In FIGS. 2A and 3A, that angle is about 30 degrees and the overall length L of the surgical clip 34 is about 0.25 inches to about 1.0 inches. In FIGS. 2B and 3B, the surgical clip 62 has an angle A of about 45 degrees and the same range of lengths as with the embodiment of FIGS. 2A and 2B. The actual angle may be any angle smaller than an orthogonal angle and preferably, the angle A may range from about 30 degrees to about 60 degrees, While the clamping surface regions 58, 60 are shown having a curvature, they, as well as the other regions of the longitudinally extending members 36, 38 may also be straight in configuration and still be within the purpose and intent of the present invention.

In FIGS. 2C and 3C, the angle A is again about 30 degrees with the surgical clip 64 having a greater length, that is, within the range of about 1.0 inches to about 2.0 inches and in FIG. 2D and 3D the angle A of the surgical clip 66 is about 45 degrees and the length is within the same range as the embodiment of FIGS. 2C and 3C. Accordingly, as now can be understood, in each of the surgical clips 34, 62, 64, and 66 the clamping surface regions 58, 60 extend from the first regions 54, 56 at angles smaller than orthogonal angles and there are differing lengths that are available to the surgeon in occluding a particular blood vessel. In addition the angles A of the various lips can be between about 10 degrees to 60 degrees, and preferably between about 45 and 60 degrees.

As such, the surgeon has a selection of surgical clips to use in any particular procedure depending upon the size and location of the blood vessel that is desired to be occluded. As a further option in the selection of a particular surgical clip, taking the surgical clip 34 of FIGS. 2A and 3A as an example, the amount of force tending to close the longitudinally extending members 36, 38, that is, the squeezing force or pressure applied against a blood vessel may also be varied.

To change the amount of force, the coiled spring 48 can be constructed by a number of differing ways. One means would be to alter the coiled spring 48 to make it more robust such as by increasing its size or be making it out of a stiffer material such that the spring bias is increased. As an alternative method, the cross-section of the clip geometry can be modified to minimize contact stresses at the clamping surface regions where the vessel is clamped, that is, the actual point of contact with the vessel can be rounded, sharp edged or some configuration that varies in between. Also a protective sleeve can be added or the surgical clip can be manufactured out of a pliable material such as a polycarbonate plastic. If a protective coating or plastic material is used, the surgical clip would then be a single use device to avoid subjecting the plastic material to multiple sterilization processes. In any event, by providing the surgeon with surgical clips having differing degrees of closing pressure, the surgeon can select the surgical clip depending upon the state or condition of the particular blood vessel that is being occluded. The variable pressure is preferred to be within 70 and 120 psi.

Turning now to FIGS. 4A, 4B and 4C, there is shown a top view, a side view and a perspective view, respectively, of a multi-vessel surgical clip 68 constructed in accordance with the present invention. As can be seen, the multi-vessel surgical clip 68 is a long L-shaped configuration having a pair of longitudinally extending members 70, 72 with distal ends 74, 76 and proximal ends 78, 80. As with the other embodiments, at the proximal ends 78, 80, there is a coiled spring 82 and a pair of actuating legs 84, 86. At or proximate to the proximal ends 78, 80, each of the longitudinally extending members 70, 72 has a first region 88, 90 extending from the coiled spring 82 in a generally straight configuration and there are also clamping surface regions 92, 94 that extend from the first regions 88, 90 and when certain vascular vessels are located in occluding those vessels during the use of the multi-vessel surgical clip 68. In this embodiment, the angle B is a relatively sharp angle and may be about 80 degrees approaching an L-shape of a ninety degree angle. The angle B, however, can be any angle ranging from about 10 degrees up to 80 degrees. In addition, the length of the multi-vessel surgical clip 68 can range from about 0.5 inches to about 2.5 inches.

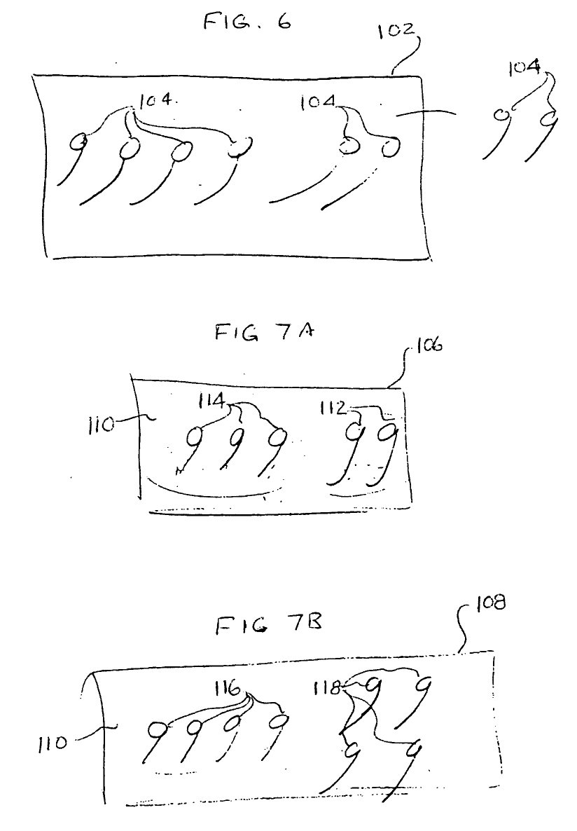

Turning now to FIG. 5, there is shown a schematic view of the multi-vessel surgical clip 68 of FIGS. 4A-4C and illustrating the use of the device. As can now be seen, the multi-vessel clip 68 actually can occlude a plurality of blood vessels identified in FIG. 5 as a major blood vessel 96, such as the carotid artery, and two smaller blood vessels 98, 100 that branch off from the major blood vessel 96. As such the major blood vessel 96 is occluded by being squeezed within the first regions 88, 90 and the smaller blood vessels 98, 100 are occluded by being squeezed within the mating clamping surface regions 92, 94. Thus, by the particular length and configuration of the multi-vessel surgical clip 68, the surgeon can occlude all three vessels and avoid the need for three separate surgical clips as is currently used in such procedure. The particular procedure for which the multi-vessel surgical clip 68 is intended is for the occluding of a carotid artery and, to aid in the clamping pressure and effectiveness, there may be a plastic or rubber sleeve or covering over the multi-vessel surgical clip 68.

Turning now to FIG. 6 there is shown a standard surgical clip set 102 that is conventionally used for various surgical procedures to provide individual surgical clips 104 to the surgeon. In the conventional surgical clip set 102, there are a plurality of surgical clips 104 of differing sizes in order to provide all of the necessary tools to the surgeon, without regard to the particular surgical procedure and the need for very specific surgical clips for a specific surgical procedure. Therefore, at the present, the surgical clip set 102 is generally a cardboard backing on which the various surgical clips 104 are mounted.

Turning now to FIGS., 7A and 7B, there is shown surgical clip sets 106, 108, respectively, arranged in accordance with the present invention and where the specific surgical clips are predetermined in accordance with the particular procedure being carried out. The surgical clips are carried by a container, such as a tray 110, and within which there may be receptacles formed therein by a pliable material such as Styrofoam into which each surgical clip is positioned. Thus, in each tray 110, there is a predetermined plurality of the surgical clips and, in each case, the quantity and type of surgical clips are pre-selected in accordance with a corresponding particular surgical procedure and the clips can be of the type previously described herein.

As examples, the surgical clip set 106 as shown in FIG. 7A can have at least two surgical clips 112 of a predetermined size and at least three other surgical clips 114 of a smaller size than the predetermined size of the surgical clips 112. Such a set is particularly suited for a surgical procedure known as a carotid endarectomy and thus, the surgeon has a customized set of a predetermined, selected quantity and type of surgical clips available for the operation and need not select from a large quantity of surgical clips where many may simply not even be suitable for the particular operation, thereby reducing the time for selection and easing the selection process of the surgeon who is already fully occupied with the surgical procedure.

As another example, with the surgical clip set 108 of FIG. 7B, there can be at least four surgical clips 116 of a predetermined size and at least four other surgical clips 118 of a smaller size than the predetermined size of the surgical clips 116. Such a surgical clip set is particularly suited for a surgical procedure known as a femoral popliteal bypass and thus, again, the surgeon has a customized set of a predetermined, selected quantity and type of surgical clips available for the operation.

It should be noted that the aforesaid examples are merely two of many surgical clip sets that can be provided to the surgeon with a predetermined plurality of surgical clips of differing sizes and quantities that are pre-selected for a particular operation so that the surgeon has a convenient and customized set of surgical clips that are specially predetermined for a specific surgical procedure.

Thus the surgical clips shown and described in the present specification can by used for a number of procedures and among those procedures are the following:
1. carotid endarectomy
2. endarectomy of the upper or lower limb arteries
3. femoral-popliteal bypass
4. aorta-renal bypass
5. aorta-iliac-femoral bypass
6. embolectomy, thrombectomy of the carotid artery or jugular vein
7. embolectomy, thrombectomy of the upper or lower limb vessels
8. resection of the carotid artery or jugular vein with anastomosis
9. resection of the upper or lower limb vessels with anastomosis
10. angiectomy of the carotid artery or jugular vein
11. angiectomy of the upper or lower limb arteries of veins
12. arteriovenostomy for renal dialysis
13. revision or removal of arteriovenous shunt for renal dialysis
14. reimplantation of aberrant renal vessel
15. trauma surgery.

Other procedures, not listed herein may, of course, be carried out with the aid of one of more of the vascular clips described herein.

An additional feature of the invention can be with the use of color coding of the individual surgical clips or sets of clips, that is, the surgical clips can be color coded in accordance with one or more of their closing pressure, size, type and use. Thus, a surgical clip having a particular closing pressure can therefore be a common color to readily identify to the surgeon that closing pressure and the same color coding can also be used to identify the size of a particular clip such as having all small clips of the same color so that the surgeon can quickly identify the desired surgical clip. The color coding principle can also be applied to sets of surgical clips where a set, for example, of surgical clips to be used for a carotid endarectomy could all be the same color so that the surgeon could immediately recognize the set of surgical clips intended and customized for that specific procedure. It should be readily understood that reference to color coding of a surgical clip is meant to cover including such color on at least a portion of the surgical clip.

Those skilled in the art will readily recognize numerous adaptations and modifications which can be made to the surgical clips and methods of the present invention which will result in an improved surgical clip and method, yet all of which will fall within the scope and spirit of the present invention as defined in the following claims. Accordingly, the invention is to be limited only by the following claims and their equivalents.

What is claimed is:

1. A surgical clip comprising:
a pair of longitudinally extending members having respective first regions and mating clamping surface regions;
a biasing element joined to said first regions to resiliently bias said mating clamping surface regions of said longitudinally extending members toward engagement; and
said member clamping surface regions extending relative to said first regions at a non-orthogonal offset angle.

2. The surgical clip of claim 1 wherein said biasing element is capable of providing a variable clamping force between said clamping surfaces.

3. The surgical clip of claim 2 wherein said variable clamping force is a range between 70 and 120 psi.

4. The surgical clip of claim 1 wherein said offset angle is between approximately 30 and 60 degrees.

5. The surgical clip of claim 1 wherein said longitudinally extending members mating clamping surface regions are curved.

6. A surgical clip comprising:
a pair of longitudinal extending members having respective first regions and at least two mating clamping surface regions;
a biasing element coupled to said member first regions to resiliently bias said mating clamping surface regions of said longitudinally extending members toward respective engagements, and
each of said mating clamping surface regions extending relative to said first regions at non-orthogonal angles.

7. The surgical clip of claim 6 wherein the clamping force is different between the respective mating clamping surface regions.

8. The surgical clip of claim 6 wherein said biasing element is capable of providing a variable clamping force between said clamping surfaces.

9. The surgical clip of claim 8 wherein said variable clamping force is a range between 70 and 120 psi.

10. The surgical clip of claim 6 wherein said offset angle is between approximately 30 and 60 degrees.

11. The surgical clip of claim 6 wherein said at least one of said clamping surface regions are curved.

12. A surgical clip system comprising:
a container having receptacles organized thereon for receipt of a plurality of surgical clips;
a predetermined plurality of surgical clips disposed within said receptacles, said plurality of clips include clips of at least one predetermined type and number in accordance with a corresponding particular surgical procedure, at least one of the clips comprises:
a pair of longitudinally extending members having respective first regions and mating clamping surface regions;
a biasing element joined to said first member regions to resiliently bias said mating clamping surface regions of said longitudinally extending members toward engagement; and
said clamping surface regions extending relative to said member first regions at a non-orthogonal offset angle.

13. The system of claim 12 wherein at least one other clip comprises:
a pair of longitudinally extending members having respective first regions and at least two mating clamping surface regions;
a biasing element coupled to said first regions to resiliently bias said mating clamping surface regions of said longitudinally extending members toward respective engagements; and
each of said mating clamping surface regions extending relative to said first regions at non-orthogonal offset angles.

14. The system of claim 12 wherein said receptacles provide an organization of said clips in said container that corresponds to their use during said surgical procedure.

15. The system of claim 12 wherein said surgical procedure is a carotid endarectomy.

16. The system of claim 15 wherein said plurality of clips include at least two clips of a particular size and at least three other clips of at least one smaller size.

17. The system of claim 12 wherein said surgical procedure is a femoral popliteal bypass.

18. The system of claim 17 wherein said plurality of clips include a least four clips of a particular size and at least four other clips of at least one smaller size.

19. A method for a surgical clip system comprising the steps of:
determining at least one type of surgical clips desired for a particular surgical procedure;
determining corresponding sizes and numbers of the surgical clips types desired for said particular surgical procedure; and
arranging said clips on a container having receptacles organized thereon for receipt of identified surgical clips for ready use by a surgeon for performing the surgical procedure.

20. The method of claim 18 wherein said particular surgical procedure is a carotid endarectomy.

21. The method of claim 20 wherein said determining steps include determining at least two clips of a particular size and at least three other clips of at least one smaller size.

22. The method of claim 18 wherein said particular surgical procedure is a femoral popliteal bypass.

23. The method of claim 22 wherein said determining steps include determining at least four clips of a particular size and at least four other clips of at least one smaller size.

24. The method of claim 18 wherein said determined surgical clip type includes a clip having
a pair of longitudinally extending members having respective first regions and mating clamping surface regions;
a biasing element joined to said first regions to resiliently bias said mating clamping surface regions of said longitudinally extending members toward engagement; and
said member clamping surface regions extending relative to said first regions at a non-orthogonal offset angle.

25. The method of claim 18 where said determined surgical clip type includes a clip having;
a pair of longitudinally extending members having respective first regions and at least two mating clamping surface regions;
a biasing element coupled to said member first regions to resiliently bias said mating clamping surface regions of said longitudinally extending members toward respective engagements; and
each of said mating clamping surface regions extending relative to said first regions at non-orthogonal offset angles.

26. A color coding system for use with a plurality of surgical clips having differing characteristics, said system comprises a color coding applied to surgical clips having a common characteristic.

27. The color coding system as defined in claim 26 wherein the common characteristic is closing pressure of a surgical clip.

28. The color coding system as defined in claim 26 wherein the common characteristic is size of a surgical clip.

29. The color coding system as defined in claim 26 wherein the common characteristic is the use of a surgical clip.

30. The color coding system as defined in claim 26 wherein the color is disposed on at least a portion of a surgical clip.
